(19) United States
(12) Patent Application Publication
(10) Pub. No.: US 2025/0177498 A1
(43) Pub. Date: Jun. 5, 2025

(54) A DRUG FOR USE IN THE TREATMENT OF MUSCLE SPASTICITY BY CHEMICAL MUSCLE DENERVATION

(71) Applicant: BOGAZICI UNIVERSITESI, Besiktas/Istanbul (TR)

(72) Inventors: Can Ali YUCESOY, Besiktas/Istanbul (TR); Cemre Su KAYA KELES, Stuttgart (DE)

(21) Appl. No.: 18/845,257

(22) PCT Filed: Mar. 8, 2023
(86) PCT No.: PCT/TR2023/050226

§ 371 Date: Sep. 9, 2024

(30) Foreign Application Priority Data
Mar. 8, 2022 (TR) 2022/003435

(51) Int. Cl. A61K 38/48; A61K 38/45; A61P 25/28

(52) U.S. Cl. CPC A61K 38/4893; A61K 38/45; A61P 25/28

(57) ABSTRACT

The present invention relates to a drug containing botulinum toxin type A (BTX-A) and C3 transferase to be used in the treatment of muscle spasticity in cerebral palsy by chemical denervation method. While the structural and mechanical behavioral effects of BTX-A for the exposed muscles are contrary to the treatment goals; with its use together with C3 transferase, the treatment of spasticity, in which the negative effects on muscle structure and muscle mechanical behavior caused by BTX-A can be prevented, is effectively provided.

### TECHNICAL FIELD OF THE INVENTION

The present invention relates to a combined drug containing both botulinum toxin type A (BTX-A) and C3 transferase to be used in the treatment of muscle spasticity in cerebral palsy via chemical denervation method. While the effects of BTX-A on the structure and mechanical function of muscles exposed are contrary to the goals of the treatment, the treatment is effectively provided and the problems caused by BTX-A are avoided if BTX-A is used in combination with C3 transferase.

### STATE OF THE ART

Cerebral palsy is a neurological disease and is a non-progressive permanent disorder of movement and posture due to damage to the developing brain. The symptoms of cerebral palsy vary, depending on which part of the brain and how widespread the injury is. There are several factors that cause cerebral palsy that may lead problems with brain development, including:
Maternal infections affecting the developing fetus,
Inherited gene mutations that cause abnormal development,
Infant infections that cause inflammation in or around the brain
Head injury due to an accident or fall,
Fetal stroke, i.e. impaired blood flow to the developing brain,
Cerebral hemorrhage in the womb or as a newborn,
Sudden asphyxia is a lack of oxygen to the brain due to a difficult birth.

Cerebral palsy leads to spasticity in the patients' muscles characterized by high tone and exaggerated stretch reflex [1], which occurs in response to the lesion in the central nervous system [2]. The neurological problem in spasticity is due to the exaggerated and uncontrolled stretch reflex. This pathological condition leads to increased passive resistance (contracture) of the muscle. Also, spastic muscles tend to show active resistance to movement of the joint. Thus, they cause a narrowing of the range of motion of the joint and these effects of spasticity make it very difficult for the patient to move.

The commonly used method for the treatment of spasticity is BTX-A injection, which provides a chemical muscle denervation [3]. Chemical muscle denervation blocks the neuromuscular junction by injecting drugs into the so-called motor endpoints, which activate the muscle. The expected therapeutic effects of such spasticity management using BTX-A in order to reduce the pathological conditions of the patients are (i) reduction of passive resistance in the joint [4] and (ii) widening of the range of motion of the joint [5]. BTX-A, being a toxin, partially paralyzes the muscles by preventing acetylcholine-containing vesicles from draining into the synaptic cleft, thereby preventing the transmission of nerve impulses to the muscle fibers at the neuromuscular junction. [6-11]. The resulting temporary muscle denervation helps to control the exaggerated stretch reflex and reduce the neurologic pathology in spasticity by blocking stretch receptors, namely muscle spindles. BTX-A succeeds in this effect.

Despite that, the muscles exposed to BTX-A are still the main motor for joint movement, and hence, their mechanical function is extremely important. The typical mechanical effect of BTX-A, which is well known in the field, is the reduction of the active forces of the muscles exposed as would be expected from a partial paralysis [12,13]. However, research directly studying specifically BTX-A effects on muscle mechanics showed that expected benefits (i) and (ii) indicated above are lacking. Therefore, there are deficiencies in the existing knowledge of the effects of BTX-A on the structure and mechanical behavior of the muscles.

Regarding the specific experimental studies assessing plausibility of (i): it has been shown that passive force of muscles exposed to BTX-A, measured both acutely (5 days after injection) [14-18] and long-term (1 month after injection) [19], increased significantly (up to several times) compared to the control group. Accordingly, BTX-A does not decrease the passive resistance of the muscle, but rather increases it. On the other hand, regarding specific experimental studies assessing plausibility of (ii): it has been shown that BTX-A either does not change [14, 17] or in contrast, narrows [15,16] the range of active muscle force production acutely and only narrows it in the long-term [19]. The length range of active muscle force production indicates how wide/narrow the joint angle range the muscle can contribute to the range of motion of the joint. Accordingly, BTX-A does not affect the mechanics of the muscle in the direction of widening of the joint range of motion, but rather leads to narrowing of it.

On the other hand, these specific studies also point to changes in the structure of the muscle. Although BTX-A is known to reduce muscle mass [20,21], it has been demonstrated by our group that it specifically modifies the extracellular matrix and this is associated with increased passive forces of the muscle. Experimental studies have shown that BTX-A increases the collagen content of the exposed muscle in the acute period [15,16], and this effect is also valid in the long-term [19]. In addition, computational mechanics studies have also shown that these structural changes are central to the cause of narrowing of length range of active muscle force production: in the simulations, the experimentally shown increased collagen content effect of BTX-A is represented by an increased extracellular matrix mechanical stiffness [22,23]. Indeed, it is a fact that increased extracellular matrix mechanical stiffness is also evidenced by the increasing slope of the passive force-muscle length curve in experimental passive muscle force findings.

When the state of the art is evaluated, it was observed that while BTX-A, which is widely used in the treatment of spasticity [22,23] and has a budget of 4-5 billion USD worldwide, has a positive effect on neurological pathology, it negatively affects the exposed muscles, which are still the motor for movement, such that BTX-A effects are opposite to the expected therapeutic effects. Based on these findings, it is crucial to control the negative effects of BTX-A on muscle structure and mechanical behavior.

C3 transferase, like BTX-A, is an inhibitor produced by the bacteria Clostridium botulinum. The effects of C3 transferase on skeletal muscle mechanics have not been investigated before. This may be due to the fact that C3 transferase is not a denervation drug and more importantly, the correlation between the mechanical function of the muscle and muscular connective tissue structures is not widely taken into account clinically. Very limited research available on the other hand has shown that C3 transferase can modify the structural alteration of connective tissues [24].

Note that: (1) For BTX-A free muscle, current novel muscle mechanics paradigms indicate that the amplitude of force produced by the activated muscle and its length range of active force production are variable, which variation is determined by the mechanical interaction between muscle fibers and muscular connective tissues [25].

Therefore, the structure and mechanical properties of muscular connective tissues play a key role in not just the passive resistance of the muscle but also in its active mechanical behavior (i.e., muscles effects on the joint motion).

For muscle exposed to BTX-A, the state of the art shows that due to an increase in the amount of collagen in the muscular connective tissues not only the muscle's passive resistance increases, but also its active mechanical behavior changes and this leads to a narrowing of the muscle's length range of active force production. Therefore, it is necessary to develop a drug that minimizes these adverse effects, which can then be used in the treatment of spasticity in cerebral palsy patients by chemical denervation.

### Brief Description and Objects of the Invention

The present invention discloses the use of botulinum toxin type A (BTX-A) and C3 transferase and the drug used here in the treatment of muscle spasticity in cerebral palsy by chemical denervation method.

As shown in FIG. 1, BTX-A increases the amount of collagen in the exposed muscle, increases its passive force, and decreases muscle length range of active force production. These findings show that the passive resistance of the muscle exposed to BTX-A is increased and its contribution to the joint range of motion is limited, and these effects are contrary to the goals of BTX-A therapy. If the two groups in FIG. 1 are compared, (i) collagen amounts (right panel) and (ii) passive forces (left panel) of the BTX-A group were statistically and significantly higher, and (iii) the muscle length range of active force production was narrower for the BTX-A group. In the present invention, these counter-indicated effects shown in FIG. 1 are minimized.

One objective of the present invention is to provide a treatment for spasticity in cerebral palsy. By means of the combined use of BTX-A and C3 transferase, in the present invention, spasticity treatment is provided in cerebral palsy by leading to an active muscle force reduction.

Another objective of the present invention is to prevent the occurrence of the above described muscle mechanics problems, while still providing control over the neurological problem in the treatment of cerebral palsy with chemical denervation. In the present invention, by means of the combined use of BTX-A and C3 transferase, and compared to the standard use of BTX-A alone, this invention does minimize (i) the increase in the collagen content, (ii) the increase in the muscle's passive resistance and (iii) the narrowing of the muscle's length range of active force production for the muscle exposed to chemical denervation.

### DESCRIPTION OF THE FIGURES

FIG. 1: A) muscle forces and B) amounts of collagen for the control group compared to the BTX-A injected group.

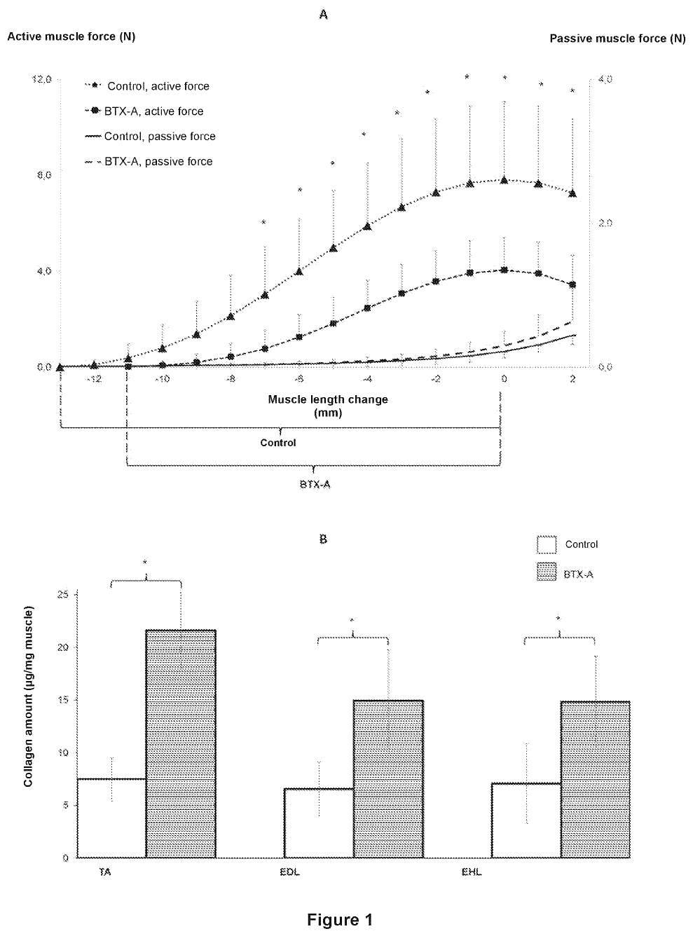

FIG. 2: For the control group compared to the C3 transferase injected group, A) Muscle forces for the control group compared to the BTX-A+C3 transferase injected group, and B) amounts of collagen.

### DETAILED DESCRIPTION OF THE INVENTION

The present invention relates to the combined use of botulinum toxin type A (BTX-A) and C3 transferase and a drug for use in the treatment of muscle spasticity in cerebral palsy via chemical denervation method. The drug used in the present invention comprises BTX-A solution containing 0.1 U of BTX-A and C3 transferase solution containing 2.5 ug C3 transferase in 1:1 volume. Both BTX-A and C3 transferase solutions are prepared with saline. BTX-A and C3 transferase are reconstituted with saline and then they are mixed for injection.

Method of preparing a drug for use in the treatment of muscle spasticity by chemical denervation in cerebral palsy, comprises the following process steps of:
i) obtaining 100 U of BTX-A solution in 4000 ul by diluting and mixing a vial of BTX-A containing 100 U of botulinum toxin type A (BTX-A) with 4 ml of saline,
ii) Obtaining a botulinum toxin type A (BTX-A) saline solution with 0.1 U of BTX-A in every 10 ul of the syringe by injecting 160 μl (4 U) of BTX-A solution and mixing it with an additional 240 μl of saline,
iii) Obtaining 25 μg of C3 transferase solution in 100 μl, thus C3 transferase saline solution with 2.5 μg C3 transferase per 10 μl in the syringe by diluting and mixing an eppendorf tube with 25 μg of C3 transferase with 100 μl of saline for C3 transferase,
iv) Taking equal volumes of samples from the syringe containing the botulinum toxin type A (BTX-A) saline solution and from the syringe containing the C3 transferase saline solution and mixing them in eppendorf.

In the present invention, the dilution is performed with saline prior to injection, separately and respectively for BTX-A and C3 transferase, with a total of 0.1 U of BTX-A and 2.5 μg of C3 transferase per 10 μl. Then, equal volumes of samples (for example, 100 μl of BTX-A and C3 transferase mixtures each) are taken from these two injectors and mixed in a new eppendorf. 20 μl intramuscular injection of the obtained drug containing BTX-A and C3 transferase was made into the target muscles of the animals at once.

For the determination of the injection site; after a mild sedation of rats with intraperitoneal ketamine (1 mg/kg), a circular area with a radius of approximately 15 mm was shaved from the center of the patella. The tibialis anterior (TA) muscle was localized by palpation with the ankle in maximal plantar flexion and the knee angle at 90°. After the center of the patella was marked, a second mark was placed along the tibia 10 mm away distally. The injection location is 5 mm lateral to the second mark and the TA muscle (along the line segment drawn between the two marks). All injections were made to a depth of 3 mm only in this vault. It is known that the depth of TA at the injection site is approximately 5-5.5 mm, and the skin thickness is approximately 0.7-1 mm. Therefore, the injections were made into the superficial half of the TA muscle.

To assess the effects of the present invention on muscle spasticity, the control group was injected with saline solution (20 ul) and the BTX-A+C3 transferase group was injected with the drug of the present invention, containing 0.1 U of BTX-A and 2.5 ug of C3 transferase in the same volume after light sedation. All injections were made one month prior to the day of the experiment and into the same point and depth in all animals consistently. Here, the Tibialis anterior (TA) muscle is the target muscle for injections. The animals were kept individually in standard cages until the day of the experiment and were free to do their normal activities. The cages were maintained in a thermally regulated animal care room with a 12-hour dark-light cycle for one month until the day of the experiment.

Male Wistar rats were divided into two groups for muscle structure analysis, with 7 samples in each group: (1) control and (2) C3 transferase (body masses 406.9±16.8 g and 437.0±33.2 g, respectively). The extracellular matrix is the connective tissue structure of the muscle that provides its passive resistance, and collagen is the main building block of this mechanical resistance. Therefore, the change in the amount of collagen in the muscle is an ideal analysis to assess the effect of BTX-A on the muscle structure. Colorimetric analysis of hydroxyproline method was used to determine the amount of collagen. This method, which has also been used for the determination of collagen content in BTX-A injected muscles [19], is based on the spectrophotometric analysis of acid-hydrolyzed tissue samples. 1 month after injections, muscle biopsies were removed immediately after euthanasia, snap frozen in liquid nitrogen, and stored at 80° C. until analysis. Muscle samples were subjected to chemical analysis steps and the amount of collagen-specific amino acid, hydroxyproline in the tissue was determined. In summary, the muscles were hydrolyzed in 5 N hydrochloric acid for 12 hours at 130° C., and afterwards, the hydrolyzate samples were oxidized with chloramine-T solution for 25 min incubation at room temperature, and impurities were separated by toluene treatment. The remaining liquid layer, which contains the hydroxyproline products, was heated in boiling water for 30 min to convert the oxidation product to pyrrole, the resulting pyrrole reaction product was removed in a second toluene extraction and the remaining solution was mixed with Ehrlich's reagent for 30 min. Thus, after completing the chemical steps, the measurement was started and the absorbance values were read at 560 nm using a UV-Visible spectrophotometer and for standard solutions (solutions with known amount of collagen) in addition to muscle samples. Here, the absorbance value characterizes the hydroxyproline contained in the muscle samples, and accordingly the quantification of the total amount of collagen is provided. As a result of the regression analysis, a mathematical relationship was obtained showing the relationship between the absorbance value and the amount of collagen. By using this relation, the amount of collagen contained in muscle samples, absorbance value of which was measured was found in terms of μg/mg muscle weight. The Shapiro-Wilk test was used to check whether the collagen amount data according to the invention were normally distributed. Accordingly, the unpaired t- or Mann-Whitney U test was used for comparisons between groups. For the control and C3 transferase groups, the amounts of collagen for the injected Tibialis anterior (TA) muscle were 7.5±2.1 and 6.1±1.2 mg collagen/mg muscle, respectively. In addition, collagen amounts were also measured for other muscles of the same compartment that were adjacent to the TA muscle, but not injected. For the extensor digitorum longus (EDL) muscle, 6.6±2.5 and 6.0±2.1 mg collagen/mg muscle were measured for the control and C3 transferase groups, respectively. For the extensor hallucis longus (EHL) muscle, 7.1±3.8 and 7.9±1.8 mg collagen/mg muscle for control and C3 transferase groups were measured, respectively. Statistical analyzes showed that injection of C3 transferase did not cause a significant change in the amount of collagen in all exposed muscles.

Male Wistar rats were divided into two groups for muscle mechanics analyses, with 7 samples in each group: (1) control and (2) BTX-A+C3 transferase (body masses 406.9±16.8 g and 414.1±27.9 g, respectively). On the day of the experiment, the animal was operated under anesthesia in such that the tendon and nerve of the target muscle were exposed and placed in the apparatus. The tendon was attached to a force transducer and the nerve was placed on the electrode. All necessary measures were taken by applying previously established and widely published methods in order to maintain the body temperature of the animal at 37° C. throughout the experiment and to maintain the physiological state of its tissues. In order to make mechanical measurements, the anterior crural compartment was exposed during the surgical preparation, the sciatic nerve was reached in order to enable muscle activation, and Kevlar threads were sewn to the distal tendon of the tibialis anterior (TA) muscle. Then, the TA tendon was attached to the force transducer with these threads, and during mechanical measurements, by changing the position of the force transducer, the muscle was lengthened, starting from a short length and bringing it to long lengths [19]. Passive forces were measured when the muscle was not stimulated and active forces were measured when the muscle's nerve was electrically stimulated. Two consecutive twitches followed by a tetanic plateau were obtained in electrical stimulation of the nerve. Passive muscle forces were calculated as the force data over the time after the second twitch ended where the muscle is inactive, while the forces at the tetanic plateau represent muscle total force in fully active state of the muscle. Active muscle forces were calculated by subtracting the measured passive muscle forces from the total muscle forces. Electrical stimulation was provided by sending a constant current and high frequency stimulus to ensure consistency between measurements, and the muscle is activated to produce the maximal force it can produce each time. The obtained muscle force-muscle length data were fitted with a polynomial function using the least squares method criteria as follows:

$$y = {a}_{0} + {a}_{1} x + {a}_{2} {x}^{2} + ⋯ + {a}_{n} {x}^{n} \qquad Formula 1$$

In formula I, y represents the isometric muscle force and x represents muscle length. a0, a1 . . . an are the coefficients determined by the fit operation. The lowest ranking of polynomials, which still makes a significant contribution to explaining changes in muscle length and muscle force data, was selected by one-way ANOVA. These polynomials were used to average muscle length-force data and to calculate standard deviations for all animals in both groups.

Passive muscle force characterizes the passive resistance that the muscle exerts on the joint. The finding that passive force of the BTX-A+C3 transferase group is (a) higher than that of the control group would mean that the addition of C3 transferase to BTX-A could not fix the problem caused by BTX-A. On the other hand, if the passive force of the BTX-A+C3 transferase group is (b) equal to that of the control group, this would show that the unwanted BTX-A effect problem has been resolved.

Muscle length range of active force production is the muscle length range between the longest muscle length at which the activated muscle produces zero force and the muscle length at which it produces its maximal force. Muscle length range of active force production therefore, characterizes the range of joint angles in which the muscle can contribute to joint motion. The finding that muscle length range of active force production of the BTX-A+C3 transferase group (a) is lower than that of the control group would mean that the problem caused by BTX-A could not be eliminated by adding C3 transferase to BTX-A. On the other hand, the finding that muscle length range of active force production of the BTX-A+C3 transferase group (b) is equal to that of the Control group would mean that the unwanted BTX-A effect problem has been resolved.

In the present invention, two-way ANOVA (factors: muscle length and animal group) was used for passive and active muscle force data. If statistically significant main effects were found, Bonferroni post-hoc tests were performed to localize within-factor differences. The Shapiro-Wilk test was used to check whether the height range data for active muscle force production were normally distributed. Accordingly, the unpaired t- or Mann-Whitney U test was used for comparisons between groups.

Statistical analyzes showed that the active force of the injected tibialis anterior (TA) muscle was reduced by 43.5% in the C3+BTX-A group compared to the control group; and that there was no significant differences between the groups in muscle passive forces and in muscle length range of active force production.

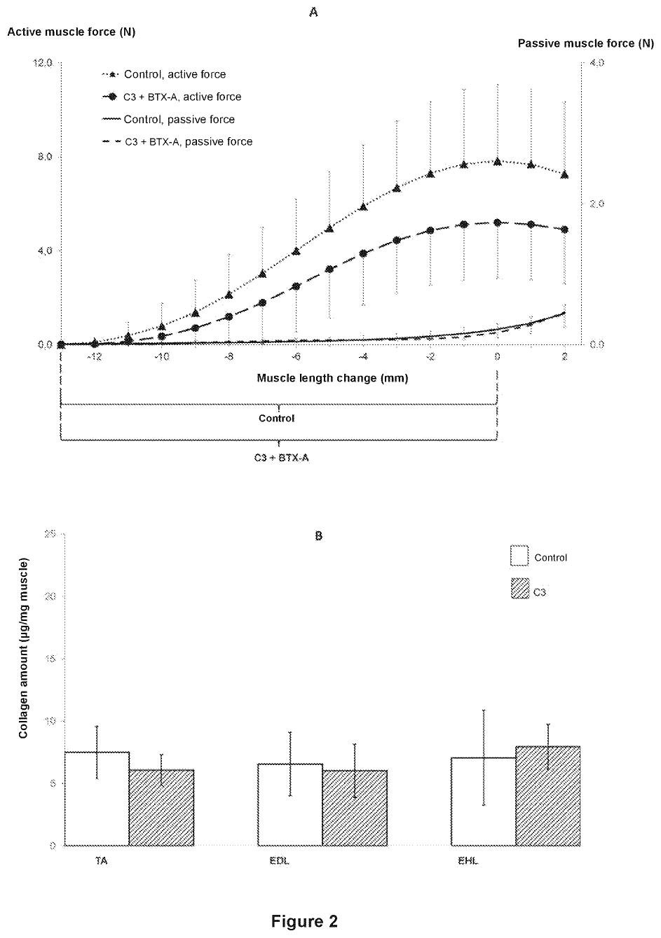

According to comparisons between groups, there was no statistically significant difference in collagen amounts between the C3 transferase group and the control group (FIG. 2, right panel). There were no statistically significant differences between BTX-A+C3 transferase and the control group for muscle passive forces and muscle length range of active force production (FIG. 2, left panel).

The lack of change in the amount of collagen in the muscles exposed to C3 transferase compared to the control group is valid not only for the targeted TA muscle, but also for EDL and EHL muscles of the compartment. On the other hand, the most important findings that show the technical effectiveness of the present invention are the results of muscle mechanics analysis. Compared to the control group, the BTX-A+C3 transferase group showed that this innovative drug causes no change in muscle passive force and in muscle length range of active force production. Accordingly, it has been shown that the adverse muscle mechanics effects contrary to the goals of the treatment of BTX-A that occur if it is injected alone are inhibited if BTX-A is injected together with C3 transferase.

### REFERENCES

[1] Cosgrove, A. P., Corry, I. S., and Graham, H. K., 1994, “Botulinum Toxin in the Management of the Lower Limb in Cerebral Palsy,” Developmental Medicine & Child Neurology, 36, pp. 386-396.
[2] Molenaers, G., van Campenhout, A., Fagard, K., de Cat, J., and Desloovere, K., 2010, “The Use of Botulinum Toxin A in Children with Cerebral Palsy, with a Focus on the Lower Limb,” Journal of Children's Orthopaedics, 4, pp. 183-195.
[3] Heinen, F., Desloovere, K., Schroeder, A. S., Berweck, S., Borggraefe, I., van Campenhout, A., Andersen, G. L., Aydin, R., Becher, J. G., Bernert, G., Caballero, I. M., Carr, L., Valayer, E. C., Desiato, M. T., Fairhurst, C., Filipetti, P., Hassink, R. I., Hustedt, U., Jozwiak, M., Kocer, S. I., Kolanowski, E., Krägeloh-Mann, I., Şehim, K., Mäenpää, H., Mall, V., McArthur, P., Morel, E., Papavassiliou, A., Pascual-Pascual, I., Pedersen, S. A., Plasschaert, F. S., Irene van der Ploeg, Remy-Neris, O., Renders, A., di Rosa, G., Steinlin, M., Tedroff, K., Valls, J. V., Viehweger, E., and Molenaers, G., 2010, “The Updated European Consensus 2009 on the Use of Botulinum Toxin for Children with Cerebral Palsy,” European Journal of Paediatric Neurology, 1, pp. 45-66.
[4] Sheean, G. L., 2001, “Botulinum Treatment of Spasticity: Why Is It so Difficult to Show a Functional Benefit?,” Current Opinion in Neurology, 14, pp. 771-76.
[5] Koman, L. A., Mooney, J. F., Smith, B. P., Walker, F., and Leon, J. M., 2000, “Botulinum Toxin Type A Neuromuscular Blockade in the Treatment of Lower Extremity Spasticity in Cerebral Palsy: A Randomized, Double-Blind, Placebo-Controlled Trial,” Journal of Pediatric Orthopaedics, 20, pp. 108-115.
[6] Blasi, J., Chapman, E. R., Link, E., Binz, T., Yamasaki, S., Camilli, P. de, Südhof, T. C., Niemann, H., and Jahn, R., 1993, “Botulinum Neurotoxin a Selectively Cleaves the Synaptic Protein SNAP-25,” Nature, 365, pp. 160-163.
[7] Brin, M. F., 1997, “Botulinum Toxin: Chemistry, Pharmacology, Toxicity, and Immunology,” Muscle & Nerve, 6, pp. 146-168.
[8] Dolly, J. O., de Paiva, A., Foran, P., Lawrence, G., Daniels-Holgate, P., and Ashton, A. C., 1994, “Probing the Process of Transmitter Release with Botulinum and Tetanus Neurotoxins,” Seminars in the Neurosciences, 6 (3), pp. 149-158.
[9] Esquenazi, A., and Mayer, N., 2001, “Botulinum Toxin for the Management of Muscle Overactivity and Spasticity after Stroke,” Current Atherosclerosis Reports, 3, pp. 295-298.
[10] Kareem, A. A., 2018, “Use of Botulinum Toxin A in Cerebral Palsy,” Cerebral Palsy—Clinical and Therapeutic Aspects, I. J. Al-Zwaini, ed., IntechOpen, pp. 95-112.
[11] Simpson, L. L., 1993, “Current Concepts on the Mechanism of Action of Clostridial Neurotoxins,” Botulinum and Tetanus Neurotoxins, B. R. DasGupta, ed., Springer U S, pp. 5-15.
[12] Grazko, M. A., Polo, K. B., and Jabbari, B., 1995, “Botulinum Toxin A for Spasticity, Muscle Spasms, and Rigidity,” Neurology, 45, pp. 712-717.
[13] Whelchel, D. D., Brehmer, T. M., Brooks, P. M., Darragh, N., and Coffield, J. A., 2004, “Molecular Targets of Botulinum Toxin at the Mammalian Neuromuscular Junction,” Movement Disorders, 19 (Suppl 8), pp. S7-S16.
[14] Ates, F., and Yucesoy, C. A., 2018, “Botulinum Toxin Type-A Affects Mechanics of Non-Injected Antagonistic Rat Muscles,” Journal of the Mechanical Behavior of Biomedical Materials, 84, pp. 208-216.
[15] Ates, F., and Yucesoy, C. A., 2014, “Effects of Botulinum Toxin Type A on Non-Injected Bi-Articular Muscle Include a Narrower Length Range of Force Exertion and Increased Passive Force,” Muscle and Nerve, 49, pp. 866-878.
[16] Yucesoy, C. A., and Ates, F., 2018, “BTX-A Has Notable Effects Contradicting Some Treatment Aims in the Rat Triceps Surae Compartment, Which Are Not Confined to the Muscles Injected,” Journal of Biomechanics, 66, pp. 78-85.
[17] Yucesoy, C. A., Emre Arikan, Ö., and Ates, F., 2012, “BTX-A Administration to the Target Muscle Affects Forces of All Muscles within an Intact Compartment and Epimuscular Myofascial Force Transmission,” Journal of Biomechanical Engineering, 134, pp. 111002:1-9.
[18] Yucesoy, C. A., Turkoğlu, A. N., Umur, S., and Ates, F., 2015, “Intact Muscle Compartment Exposed to Botulinum Toxin Type a Shows Compromised Intermuscular Mechanical Interaction,” Muscle and Nerve, 51, pp. 106-116.
[19] Kaya, C. S., Yilmaz, E. O., Akdeniz-Doğan, Z. D., and Yucesoy, C. A., 2020, “Long-Term Effects With Potential Clinical Importance of Botulinum Toxin Type-A on Mechanics of Muscles Exposed,” Frontiers in Bioengineering and Biotechnology, 8, p. 738.
[20] Koerte, I. K., Schroeder, A. S., Fietzek, U. M., Borggraefe, I., Kerscher, M., Berweck, S., Reiser, M., Ertl-Wagner, B., and Heinen, F., 2013, “Muscle Atrophy beyond the Clinical Effect after a Single Dose of Onabotulinumtoxina Injected in the Procerus Muscle: A Study with Magnetic Resonance Imaging,” Dermatologic Surgery, 39 (5), pp. 761-765.
[21] Multani, I., Manji, J., Hastings-Ison, T., Khot, A., and Graham, K., 2019, “Botulinum Toxin in the Management of Children with Cerebral Palsy,” Pediatric Drugs, 21, pp. 261-281.
[22] Aoki, K. R., 2002, “Immunologic and Other Properties of Therapeutic Botulinum Toxin Serotypes,” Scientific and Therapeutic Aspects of Botulinum Toxin, J. J. Brin M F, Mallett M, ed., Lippincott Williams and Wilkins, Philadelphia, pp. 103-113.
[23] Aoki, K. R., and Guyer, B., 2001, “Botulinum Toxin Type A and Other Botulinum Toxin Serotypes: A Comparative Review of Biochemical and Pharmacological Actions,” European Journal of Neurology, 8 (Suppl. 5), pp. 21-29.
[24] Schleip, R., Gabbiani, G., Wilke, J., Naylor, I., Hinz, B., Zorn, A., Jāger, H., Breul, R., Schreiner, S., and Klingler, W., 2019, “Fascia Is Able to Actively Contract and May Thereby Influence Musculoskeletal Dynamics: A Histochemical and Mechanographic Investigation,” Frontiers in Physiology, 10, p. 336.
[25] Yucesoy, C. A., 2010, “Epimuscular Myofascial Force Transmission Implies Novel Principles for Muscular Mechanics,” Exercise and Sport Sciences Reviews, 38 (3), pp. 128-134.
[26] Carlson, C. G., 2014, “Determination of Hydroxyproline Content as a Measure of Fibrosis in Nondystrophic and Dystrophic Skeletal Muscle,” Treat NMD [Online]. Available: http://www.treat-nmd.eu/downloads/file/sops/dmd/MDX/DMD_M.1.2.006.pdf.

1. A drug for use in the chemical denervation treatment of muscle spasticity in cerebral palsy, characterized by comprising botulinum toxin type A (BTX-A) and C3 transferase.

2. A drug according to claim 1, characterized by further comprising saline solution.

3. A drug according to claim 1, characterized in that, said botulinum toxin type A (BTX-A) and C3 transferase are in the form of solution diluted with saline, separately.

4. A drug according to claim 1, characterized in that, Botulinum toxin type A (BTX-A) solution diluted with saline and C3 transferase solution diluted with saline are 1:1 by volume.

5. A drug according to claim 1, characterized by comprising BTX-A saline solution containing 0.1 U of BTX-A and C3 transferase saline solution containing 2.5 ug of C3 transferase.

6. Method of preparing a medicament for use in the treatment of muscle spasticity by chemical denervation in cerebral palsy, characterized by comprising the process steps of;
v) obtaining 100 U of BTX-A solution in 4000 ul by diluting and mixing a vial of BTX-A containing 100 U of botulinum toxin type A (BTX-A) with 4 ml of saline,
vi) Obtaining a botulinum toxin type A (BTX-A) saline solution with 0.1 U of BTX-A in every 10 ul of the syringe by injecting 160 μl (4 U) of BTX-A solution and mixing it with an additional 240 μl of saline,
vii) Obtaining 25 μg of C3 transferase solution in 100 μl, thus C3 transferase saline solution with 2.5 μg C3 transferase per 10 μl in the syringe by diluting and mixing an eppendorf tube with 25 μg of C3 transferase with 100 μl of saline for C3 transferase,
viii) Taking equal volumes of samples from the syringe containing the botulinum toxin type A (BTX-A) saline solution and from the syringe containing the C3 transferase saline solution and mixing it in eppendorf.
